SEQUENCE LISTING (2 sequences)

SEQ ID NO: 1 - DNA, 30 bp, Artificial
  Primer 1 sequence
ggaggcgaattcatgaccgcaaccagctcc

SEQ ID NO: 2 - DNA, 27 bp, Artificial
  Primer 2 sequence
gggctgctgcagtcagtacgcggcgga

(19) United States
(12) Patent Application Publication
(10) Pub. No.: US 2006/0121586 A1
(43) Pub. Date: Jun. 8, 2006

(54) Process for producing phorenol

(76) Inventors: Tatsuo Hoshino, KAMAKURA-SHI, KANAGAWA (JP); Yutaka Setoguchi, Kanagawa-ken (JP); Sakayu Shimizu, Kyoto-fu (JP); Kazuyuki Tabata, Kanagawa-ken (JP)

Correspondence Address: Stephen M Haracz;Bryan Cave, 1290 Avenue of the Americas, New York, NY, 10104, US

(21) Appl. No.: 10/519,969

(22) PCT Filed: May 9, 2003
(86) PCT No.: PCT/EP03/04893

§ 371 Date: Sep. 30, 2005

(30) Foreign Application Priority Data
Jul. 4, 2002 (EP) 02014784.9

(51) Int. Cl. C12P 7/26; C12N 1/21

(52) U.S. Cl. 435/148; 435/252.3

(57) ABSTRACT

The present invention relates to processes for producing (4S)-4-hydroxy-2,6,6-trimethyl-2-cyclohexene-1-one (phorenol) from 2,6,6-trimethyl-2-cyclohexene-1,4-dione (ketoisophorone). The present invention also relates to products useful for producing phorenol from ketoisophorone, including microorganisms, cell-free extracts of such microorganisms, recombinant microorganisms, cell-free extracts of such recombinant microorganisms, and enzymes (e.g., levodione reductase). Processes for producing phorenol from ketoisophorone using such products are also provided.

The present invention relates to a process for producing (4S)-4-hydroxy-2,6,6-trimethyl-2-cyclohexene-1-one (hereinafter referred to as phorenol) from 2,6,6-trimethyl-2-cyclohexene-1,4-dione (hereinafter referred to as ketoisophorone). More specifically, the present invention concerns a process for producing phorenol from ketoisophorone by a specific microorganism, a cell-free extract thereof, a recombinant microorganism or cell-free extract thereof, or levodione reductase.

Phorenol is a useful chiral building block of naturally occurring optically active compounds such as zeaxanthin. There were a few methods reported until now for enantioselective production of phorenol [Tanaka et al., Tetrahedron:Asymmetry 6:1273 (1995); Kiyota et al., Tetrahedron:Asymmetry 10:3811 (1999)]. But these methods were inefficient for optical purity of the product, and require a multistep sequence. There was no biological process for the direct production of optically active phorenol.

Surprisingly, phorenol can be formed from ketoisophorone with high optical purity by a single step reaction using a microorganism which is capable of producing actinol from levodione.

An object of the present invention is to provide a process for producing phorenol from ketoisophorone comprising contacting ketoisophorone with a microorganism or cell-free extract thereof which is capable of producing actinol from levodione, and isolating the resulting phorenol from the reaction mixture.

Another object of the present invention is to provide a proceess for producing phorenol from ketoisophorone by contacting ketoisophorone with a recombinant microorganism or cell-free extract thereof which is expressing the levodione reductase gene, and isolating the resulting phorenol from the reaction mixture.

A further object of the present invention is to provide a process for producing phorenol from ketoisophorone by contacting ketoisophorone with levodione reductase which is capable of catalyzing the conversion of ketoisophorone regio- and stereoselectively to phorenol.

Examples of a microorganism capable of producing actinol from levodione include wild-type members of the genera Cellulomonas, Corynebacterium, Planococcus and Arthrobacter, as well as recombinant microorganisms.

A preferrred microorganism is selected from the group consisting of Cellulomonas sp. AKU672, Corynebacterium aquaticum AKU610, Corynebacterium aquaticum AKU611, Planococcus okeanokoites AKU152 and Arthrobacter sulfureus AKU635, or a mutant thereof These microorganisms are disclosed in EP 0982406. One the most preferred strains is Corynebacterium aquaticum AKU611.

The above strains were deposited at the National Institute of Advanced Industrial Science and Technology (AIST), Tsukuba Central 6, 1-1-1 Higashi, Tsukuba, Ibaraki 305-8566, Japan, in the name of F. Hoffmann-La Roche AG of Grenzacherstrasse 124, CH-4070 Basel, Switzerland on Aug. 4, 1998, under Budapest Treaty, with the deposit numbers FERM BP-6449 (Cellulomonas sp. AKU672), FERM BP-6447 (Corynebacterium aquaticum AKU610) and FERM BP-6448 (Corynebacterium aquaticum AKU611). Planococcus okeanokoites AKU152 and Arthrobacter sulfureus AKU635 are deposited at and are available from the Institute for Fermentation, Osaka (IFO), 17-85, Juso-honmachi 2-chome, Yodogawa-ku, Osaka, Japan, referring to the following deposit Numbers: IFO 15880 (Planococcus okeanokoites AKU152) and IFO 12678 (Arthrobactersulfureus AKU635).

Recombinant microorganisms used in the present invention can be prepared, e.g., by the method described in EP 1,122,315, disclosing genetic material such as an isolated DNA comprising a nucleotide sequence coding for an enzyme having levodione reductase activity, a polypeptide encoded by such a DNA, recombinant organisms and the like.

Either growing cell culture or resting cell culture or immobilized cell or cell-free extract, or the like, of said microorganism or the recombinant microorganism may be used for the production of phorenol. Said growing cell culture can be obtained by culturing said microorganism or the recombinant microorganism in a nutrient medium containing saccharides such as glucose or sucrose, alcohols, such as ethanol or glycerol, fatty acids, such as oleic acid and stearic acid or esters thereof, or oils, such as rapeseed oil or soybean oil, as carbon sources; ammonium sulfate, sodium nitrate, peptone, amino acids, corn steep liquor, bran, yeast extract and the like, as nitrogen sources; magnesium sulfate, sodium chloride, calcium carbonate, potassium monohydrogen phosphate, potassium dihydrogen phosphate, and the like, as inorganic salt sources; and malt extract, meat extract, and the like, as other nutrient sources.

Cultivation of the microorganism can be carried out aerobically or anaerobically at pH values from 4.0 to 9.0, at a temperature range from 10 to 50° C. for 15 minutes to 72 hours, preferably, at pH values from 5.0 to 8.0, at a temperature range from 20 to 40° C. for 30 minutes to 48 hours. Appropriate mixing of the culture during the cultivation will be preferable for the cell growth or the reaction.

Using the growing cell culture thus obtained, said resting cell culture or immobilized cell or cell-free extract may be prepared by any means generally known in the art.

The concentration of ketoisophorone in a reaction mixture can vary depending on other reaction conditions, but, in general, is between 0.1 g/l and 300 g/l, preferably between 1 g/l and 30 g/l.

In the present invention, phorenol can also be produced by contacting ketoisophorone with levodione reductase.

One of the most preferred levodione reductases and a method for its preparation are described in EP 1,026,235. The physico-chemical properties of this levodione reductase can be summarized as follows:
1) The levodione reductase catalyzes regio- and stereoselective reduction of levodione to actinol.
2) The relative molecular mass of the enzyme is estimated to be 142,000 to 155,000±10,000 Da, consisting of four homologous subunits having a molecular mass of 36,000±5,000 Da.
3) The optimum temperature is 15-20° C. at pH 7.0 and the optimum pH is 7.5.
4) The enzyme requires NAD⁺ or NADH as a cofactor and is highly activated by monovalent cations, such as K⁺, Na⁺, Cs⁺, Rb⁺, and NH⁴⁺.

Levodione reductase catalyzes the reduction of ketoisophorone to phorenol in the presence of a co-factor according to the following formula:
Ketoisophorone+NADH [symbol] Phorenol+NAD

For example, the standard enzyme reaction is performed as follows: The basal reaction mixture (total volume: 1 ml): 200 μl of 1 M potassium phosphate buffer (pH 7.0), 40 μl of 8 mM NADH in 0.2 mM KOH, 200 μl of ketoisophorone solution, and 20-80 μl of the enzyme solution, and water to add up to 1 ml, is incubated at pH values of from 4.0 to 9.0, at a temperature range from 10 to 50° C. for 5 minutes to 48 hours, preferably at pH values of from 5.0 to 8.0, at a temperature range from 20 to 40° C. for 15 minutes to 24 hours. Appropriate mixing of the reaction mixture will be preferable for the reaction.

The concentration of ketoisophorone in a reaction mixture can vary depending on other reaction conditions, but, in general, is between 0.1 g/l and 300 g/l, preferably between 1 g/l and 30 g/l.

Phorenol produced biologically or enzymatically in the reaction mixture as described above is extracted by an organic solvent such as ethyl acetate, n-hexane, toluene, or n-butyl to recover the phorenol into the organic solvent layer. The extract is analyzed by known method such as gas chromatography, high performance liquid chromatography, thin layer chromatography or paper chromatography, or the like. In case of the gas chromatography, the following conditions can be applied as an one of the embodiment:
Column: ULBON HR-20M (Shinwa, Japan) 0.25 mmφ+30 m
Column temperature: 160° C. (constant)
Injector temperature: 250° C.
Carrier gas: He (ca. 1 ml/min)

After the reaction, phorenol in the reaction mixture may be recovered, for example, by extraction with a water-immiscible organic solvent which readily solubilizes phorenol, such as ethyl acetate, n-hexane, toluene or n-butyl acetate. Further purification of phorenol can be effected by concentrating the extract to directly crystallize phorenol or by the combination of various kinds of chromatography, for example, thin layer chromatography, adsorption chromatography, ion-exchange chromatography, gel filtration chromatography or high performance liquid chromatography.

The following Examples further illustrate the present invention.

### EXAMPLE 1

### Phorenol Production Using Corynebacterium aquaticum AKU611 (FERM BP-6448)

Corynebacterium aquaticum AKU611 (FERM BP-6448) was inoculated into seed medium (100 mL in a 500 ml flask) containing 1.0 g/L of yeast extract, 15.0 g/L of Bacto-peptone (Difco laboratories, U.S.A), 0.2 g/L of MgSO₄.7H₂O, 3.0 g/L of K₂HPO₄, 2.0 g/L of NaCl and 22.4 g/L of glucose.H₂O, and cultivated at 30° C. with rotary shaking for 24 hours. A portion of the seed culture (100 ml) was inoculated into production medium (3.0 L in a 5-L scale jar fermentor; Type MJ-5-6, L.E.Marubishi, Japan) containing 8.0 g/L of yeast extract, 0.2 g/L of MgSO₄.7H₂O, 0.01 g/L of MnSO₄.4-5H₂O, 2.0 g/L of NaCl and 11.1 g/L of glucose.H₂O. Cultivation was conducted at 30° C. with agitation of 600 r.p.m. and aeration of 1.0 vvm. pH was maintained at 7.0 by using ammonium solution. After about 9 hours cultivation, glucose feeding was started with feeding rate of 20 g/hour. After 24 hours from the beginning of the cultivation, each of the conditions, temperature, agitation, aeration, pH, and feeding rate of glucose was shifted to 25° C., 200 r.p.m., 0.17 vvm, 6.0, and 10 g/hour, respectively, and cultivation was continued for another 24 hours. During this period, 42 g of ketoisophorone was added to the medium in four times (for example, 20 g at the beginning, 10 g at the 3^{rd} hour, 7 g at the 6^{th} hour, and 5 g at the 9^{th} hour of this period). A portion of the cultivated broth was extracted by ethylacetate to recover the phorenol into ethylacetate layer. The extract was analyzed by gas chromatography [column: ULBON HR-20M (Shinwa, Japan) 0.25 mmφ×30 m, column temperature: 160° C. (constant), injector temperature: 250° C., carrier gas: He (ca. 1 ml/min)]. As a result, 8.0 g/L of phorenol (91% conversion of ketoisophorone used) was produced. Optical purity of the product was analyzed to be 96.0% (e.e.) by gas chromatography using a chiral capillary column, BGB-176 (BGB Analytik AG, Switzerland).

### EXAMPLE 2

### Cloning of levodione Reductase Gene from Genomic DNA of Corynebacterium aquaticum AKU611 (FERM BP-6448)

Genomic DNA of Corynebacterium aquaticum AKU611 (FERM BP-6448) was prepared using Genome Isolation Kit (BIO101). Using the prepared genomic DNA as template, a complete coding sequence for the levodione reductase gene without excessive flanking region was obtained by PCR amplification using a thermal cycler (Perkin elmer 2400, U.S.A.). The two synthetic primers used were as follows:

(SEQ ID NO:1)

LV-ORF(+)
(5′-GGAGGCGAATTCATGACCGCAACCAGCTCC-3′)

(the underlined sequence is the position of an EcoRI site)

| | |
|---|---|
| LV-ORF(−) | |
| (5′-GGGCTGCTGCAGTCAGTACGCGGCGGA-3′) | (SEQ ID NO:2) |

(the underlined sequence is the position of an PstI site)

The PCR mixture (0.02 ml) contained 5 pmol of each primer, 0.2 mM of each dNTP, and 1 U of LA Taq (Takara Shuzo co.LTD/Kyoto, Japan). The initial template denaturation step consisted of 1 min at 94° C. An amplification cycle of 20 sec at 98° C., 2 min at 70° C. and 4 min at 72° C. was repeated for 25 times.

By this reaction, a DNA fragment containing a complete ORF of the levodione reductase gene (0.8 Kb) was amplified. This amplified levodione reductase gene was treated with EcoRI and PstI, and ligated with a vector, pKK223-3 (Amersham Bioscience/Buckinghamshire, England) that was predigested with EcoRI and PstI to construct a plasmid, pKKLR(1-15). E. coli JM109 was transformed with the ligation mixture, and several clones were selected for sequence analysis. The sequence of the cloned levodione reductase gene of each candidate clone was examined. One of the clones that showed completely the same sequence as the levodione reductase sequence of Corynebacterium aquaticum AKU611 (FERM BP-6448) was named as JM109[pKKLR(1-15)], and used for further experiments.

### EXAMPLE 3

### Phorenol Production Using E. coli JM109[pKKLR(1-15)]

E. coli JM109[pKKLR(1-15)] was inoculated into seed medium (100 mL in a 500 ml flask) containing 5.0 g/L of yeast extract, 10.0 g/L of Bacto-tryptone (Difco laboratories, U.S.A), 10.0 g/L of NaCl, 11.1 g/L of glucose.H₂O and 50 mg/L of Na-ampicillin (Sigma Chemical Co., USA), and cultivated at 30° C. with rotary shaking for 16 hours. A portion of the seed culture (100 ml) was inoculated into production medium (3.0 L in a 5-L scale jar fermentor; Type MJ-5-6, L.E.Marubishi, Japan) containing 7.5 g/L of K₂HPO₄, 1.9 g/L of citric acid, 0.3 g/L of ammonium iron(III) citrate, 0.49 g/L of MgSO₄.7H₂O, 22.2 g/L of glucose.H₂O, and trace elements including, for example, (NH₄)₆(Mo₇O₂₄).4H₂O, ZnSO₄.7H₂O, H₃BO₃, CuSO₄.5H₂O, MnCl₂.4H₂O, and the like. Cultivation was conducted at 30° C. with agitation of 600 r.p.m. and aeration of 1.0 vvm. pH was maintained not to dip from 6.0 by using ammonium solution. After about 9 hours cultivation, glucose feeding was started with feeding rate of 3.5 g/hour. After 24 hours from the beginning of the cultivation, each of the conditions, temperature, agitation, aeration, and feeding rate of glucose was shifted to 25° C., 200 r.p.m., 0.17 vvm, and 2.0 g/hour, respectively, and cultivation was continued for another 24 hours. During this period, 25 g of ketoisophorone was added to the medium in five times (for example, 5 g each at certain intervals). A portion of the cultivated broth was extracted by ethylacetate to recover the phorenol into ethylacetate layer. The extract was analyzed by gas chromatography as described in Example 1. As a result, 5.6 g/L of phorenol (71% conversion of ketoisophorone used) was produced. Optical purity of the product was analyzed to be 85.7% (e.e.) by gas chromatography using a chiral capillary column as described in Example 1.

1. A process for producing (4S)-4-hydroxy-2,6,6-trimethyl-2-cyclohexene-1-one (phoren-ol) from 2,6,6-trimethyl-2-cyclohexene-1,4-dione (ketoisophorone) comprising contacting ketoisophorone with a microorganism which is capable of producing actinol from levodione or with a cell-free extract thereof, with a recombinant microorganism which is capable of producing actinol from levodione or with a cell-free extract thereof, or with levodione reductase, and isolating the resulting phorenol from the reaction mixture.

2. A process for producing phorenol from ketoisophorone comprising contacting keto-isophorone with a microorganism which is capable of producing actinol from levodione, or with a cell-free extract thereof, and isolating the resulting phorenol from the reaction mixture.

3. A process for producing phorenol from ketoisophorone comprising contacting keto-isophorone with a microorganism or cell-free extract thereof selected from members of the genera Cellulomonas, Corynebacterium, Planococcus and Arthrobacter, which are capable of selective asymmetric reduction of levodione to actinol, and isolating the resulting phorenol from the reaction mixture.

4. The process according to claim 3, wherein the microorganism is selected from the group consisting of Cellulomonas sp. AKU672 (FERM BP-6449), Corynebacterium aquaticum AKU610 (FERM BP-6447), Corynebacterium aquaticum AKU611 (FERM BP-6448), Planococcus okeanokoites AKU152 (IFO 15880) and Arthrobacter sulfureus AKU635 (IFO 12678), and mutants thereof.

5. The process according to claim 3, wherein the microorganism is Corynebacterium aquaticum AKU611 (FERM BP-6448).

6. A process for producing phorenol from ketoisophorone by contacting ketoisophorone with a recombinant microorganism or cell-free extract thereof which is expressing the levodione reductase gene, and isolating the resulting phorenol from the reaction mixture.

7. The process according to claim 6, wherein the levodione reductase gene is derived from a microorganism belonging to the genus Corynebacterium.

8. The process according to claim 7, wherein the levodione reductase gene is derived from Corynebacterium aquaticum AKU611 (FERM BP-6448) or a functional equivalent, subculture, mutant or variant thereof.

9. A process for producing phorenol from ketoisophorone by contacting ketoisophorone with levodione reductase which is capable of catalyzing the conversion of ketoisophorone regio-and stereoselectively to phorenol.

10. The process according to claim 9, wherein the levodione reductase is derived from a microorganism belonging to the genus Corynebacterium.

11. The process according to claim 10, wherein the levodione reductase is derived from Corynebacterium aquaticum AKU611 (FERM BP-6448) or a mutant thereof.

12. The process according to claim 1, wherein the reaction is carried out at pH values of from 4.0 to 9.0, at a temperature range from 10 to 50° C. and for 15 minutes to 72 hours.

13. The process according to claim 12, wherein the reaction is carried out at pH values of from 5.0 to 8.0, at a temperature range at from 20 to 40° C. and for 30 minutes to 48 hours.

14. The process according to claim 2, wherein the reaction is carried out at pH values of from 4.0 to 9.0, at a temperature range from 10 to 50° C. and for 15 minutes to 72 hours.

15. The process according to claim 14, wherein the reaction is carried out at pH values of from 5.0 to 8.0, at a temperature range at from 20 to 40° C. and for 30 minutes to 48 hours.

16. The process according to claim 3, wherein the reaction is carried out at pH values of from 4.0 to 9.0, at a temperature range from 10 to 50° C. and for 15 minutes to 72 hours.

17. The process according to claim 16, wherein the reaction is carried out at pH values of from 5.0 to 8.0, at a temperature range at from 20 to 40° C. and for 30 minutes to 48 hours.

18. The process according to claim 6, wherein the reaction is carried out at pH values of from 4.0 to 9.0, at a temperature range from 10 to 50° C. and for 15 minutes to 72 hours.

19. The process according to claim 18, wherein the reaction is carried out at pH values of from 5.0 to 8.0, at a temperature range at from 20 to 40° C. and for 30 minutes to 48 hours.

20. The process according to claim 9, wherein the reaction is carried out at pH values of from 4.0 to 9.0, at a temperature range from 10 to 50° C. and for 15 minutes to 72 hours.

21. The process according to claim 20, wherein the reaction is carried out at pH values of from 5.0 to 8.0, at a temperature range at from 20 to 40° C. and for 30 minutes to 48 hours.
